(19) United States
(12) Patent Application Publication
(10) Pub. No.: US 2025/0223543 A1
(43) Pub. Date: Jul. 10, 2025

(54) ACTIVE COMPOSITIONS

(71) Applicants: MARLOW FOODS LIMIED, Stokesley (GB); HERIOT-WATT UNIVERSITY, Edinburgh (GB)

(72) Inventors: Muyiwa Adetokunbo AKINTOYE, Guisborough Cleveland (GB); Stephen Robert EUSTON, Edinburgh (GB); Timothy John Andrew FINNIGAN, Hutton Rudby, Yorkshire (GB); Julien LONCHAMP, Edinburgh (GB); lINDA Christina PRAVINATA, Leeds, Yorkshire (GB); Stewart John RADFORD, York, Yorkshire (GB)

(21) Appl. No.: 18/851,252

(22) PCT Filed: Mar. 30, 2023
(86) PCT No.: PCT/GB2023/050827

§ 371 Date: Sep. 26, 2024

(30) Foreign Application Priority Data
Mar. 31, 2022 (GB) 2204668.4

(51) Int. Cl. C12N 1/14; A23L 29/10; A23L 29/269; A23L 31/00

(52) U.S. Cl. CPC C12N 1/14; A23L 29/10; A23L 29/269; A23L 31/00

(57) ABSTRACT

A method of producing an active composition including (i) selecting a fermentation broth including a filamentous fungus, for example Fusarium venenatum; and (ii) removing water from said broth or a part thereof to produce a solid product, wherein said solid product is an active composition which may function as a viscosifier, gellator, foamer, foam stabiliser or emulsifier.

This invention relates, particularly although not exclusively, to an active composition and a method of producing an active composition. The invention extends to an active composition per se and methods of using the active compositions for example as rheology modifiers and/or surface active materials.

Rheology modifiers and/or surface active materials such as thickeners, gelators, foamers, foam stabilisers and emulsifiers are well-known. They often comprise man-made chemicals which include carefully selected hydrophobic and/or hydrophilic chemical functionalities. Such chemicals may have wide-ranging applications in household goods (e.g. soaps, detergent, fabric conditioner), in cosmetics (e.g. personal care, skin care, cleansers, deodorants, hair care, perfumery, sunscreens, moisturizers), in industrial goods (e.g. inks, lubricants, anti-fogging, liquids, adhesives) and in pharmaceutical, fungicides and herbicides.

Rheology modifiers and/or surface active materials may also be used in foodstuffs to enhance visual, mechanical, taste and/or mouth-feel properties of foodstuffs. Such materials may be natural or synthetic. Widely used natural functional ingredients for foodstuffs include milk and egg-derived proteins which are known to have high thickening, gelling, foaming and emulsifying properties.

However, to reduce its environmental footprint, the food industry is looking for sustainable alternatives to animal-derived functional ingredients. In addition, the industry seeks vegan-friendly ingredients, particularly as the demand for vegan foodstuffs (and other products) increases.

It is an object of the present inventions to address the above-described problems.

It is an object of the present invention to produce vegan rheology modifiers and/or surface active materials which may have wide-range food and non-food applications.

According to a first aspect of the invention, there is provided a method of producing an active composition, the method comprising:
(i) selecting a fermentation broth comprising a filamentous fungus; and
(ii) removing water from said broth or a part thereof to produce a solid product, wherein said solid product is an active composition.

Said filamentous fungus preferably comprises fungal mycelia and suitably at least 80 wt %, preferably at least 90 wt %, more preferably at least 95 wt % and, especially, at least 99 wt % of the fungal particles in said formulation comprise fungal mycelia. Some filamentous fungi may include both fungal mycelia and fruiting bodies. Said fungal particles preferably comprise a filamentous fungus of a type which does not produce fruiting bodies.

Said filamentous fungus preferably comprises fungus selected from fungi imperfecti.

Preferably, said filamentous fungus comprises, and preferably consists essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, VA.).

Filamentous fungi in said broth may comprise filaments having lengths of less than 1000 μm, preferably less than 800 μm. Said filaments may have a length greater than 50 μm, preferably greater than 100 μm. Preferably, fewer than 5 wt %, preferably substantially no, filaments in said broth have lengths of greater than 5000 μm; and preferably fewer than 5 wt %, preferably substantially no filaments have lengths of greater than 2500 μm. Preferably, values for the number average of the lengths of said filamentous fungus in said broth are also as stated above. Thus, the number average of the lengths of said filamentous fungus in said broth may be in the range 50 μm to 1000 μm, preferably in the range 50 μm to 500 μm.

Said filamentous fungus in said broth selected in step (i) may comprise filaments having diameters of less than 20 μm, preferably less than 10 μm, more preferably 5 μm or less. Said filaments may have diameters greater than 1 μm, preferably greater than 2 μm. Preferably, values for the number average of said diameters of said fungal particles in said formulation are also as stated above. Thus, the number average of said diameters of said fungal particles in said broth are preferably in the range 1 μm to 20 μm.

Said filamentous fungus in said broth selected in step (i) may comprise filaments having an aspect ratio (length/diameter) of less than 1000, preferably less than 750, more preferably less than 500, especially of 250 or less. The aspect ratio may be greater than 10, preferably greater than 40, more preferably greater than 70. Preferably, values for the average aspect ratio of said filamentous fungus (i.e. the number average of the lengths of the particles divided by the number average of the diameters of the said filamentous fungus in said broth are also as stated above. Thus, the number average of the lengths of the filaments divided by the number average of the diameters of the said filaments fungus in said broth is preferably in the range 10 to 1000, more preferably in the range 40 to 250.

In a preferred embodiment, a laser diffraction method (also referred to as “static light scattering” is used to assess particle sizes as described herein. The particle sizing method is based on the light scattering pattern, i.e., intensity vs. scattering angle of a sample. As a light beam propagates through a suspension, the scattering pattern behaves differently depending on the particle size. The obtained scattering pattern data is converted into a particle size distribution (PSD) using a mathematical model known as ‘Mie Theory’ with known refractive index of the particles and dispersion medium.

Filamentous fungi in said broth may comprise filaments having lengths of greater than 150 μm, preferably greater than 500 μm. Preferably, greater than 5 vol %, more preferably greater than 10 vol % of filamentous fungi in said broth comprise filaments having lengths of greater than 150 μm.

Preferably, fewer than 5 wt %, preferably substantially no, filaments in said broth have lengths of greater than 5000 μm; and preferably fewer than 5 wt %, preferably substantially no filaments have lengths of greater than 2500 μm.

Preferably, greater than 90 vol %, more preferably greater than 95 vol % of filamentous fungi in said broth comprise filaments having lengths of less than 2000 μm.

In said solid product, said filamentous fungi may comprise filaments having lengths of greater than 150 μm, preferably greater than 500 μm. Preferably, greater than 5 vol %, more preferably greater than 10 vol % of filamentous fungi in said solid product comprise filaments having lengths of greater than 150 μm.

Preferably, fewer than 5 wt %, preferably substantially no, filaments in said solid product have lengths of greater than 5000 μm; and preferably fewer than 5 wt %, preferably substantially no filaments in said solid product have lengths of greater than 2500 μm.

Preferably, greater than 90 vol %, more preferably greater than 95 vol % of filamentous fungi in said solid product comprise filaments having lengths of less than 2000 μm.

Said solid product may include less than 15 wt %, suitably less than 10 wt %, preferably less than 8 wt %, more preferably less than 7.5 wt % of water. Said solid product may include at least 0.01 wt %, for example at least 1 wt %, at least 2 wt % or at least 3 wt %, water.

Said solid product may include at least 35 wt %, preferably at least 40 wt %, protein on a dry matter basis. Said solid product may include less than 65 wt %, preferably less than 60 wt %, protein on a dry matter basis.

Said solid product may include at least 4 wt %, preferably at least 8 wt %, N on a dry matter basis. Said solid product may include less than 15 wt %, preferably less than 10 wt %, N on a dry matter basis.

Said solid product may include at least 10 wt %, preferably at least 15 wt %, dietary fibre on a dry matter basis. Said solid product may include less than 30 wt %, preferably less than 23 wt %, dietary fibre on a dry matter basis.

Said solid product may include at least 4 wt %, preferably at least 7 wt %, fat on a dry matter basis. Said solid product may include less than 20 wt %, preferably less than 10 wt %, fat on a dry matter basis.

It is found that certain heat treatments of the broth or a said part thereof, may affect, for example, reduce, the activity of said active composition prepared in the method. Suitably, the broth is not heated to a temperature which is greater than 75° C. or greater than 79° C. at any time prior to step (ii) of the method. Preferably, the broth is not held at a temperature which is greater than 75° C. or greater than 79° C. for more than 1 minute or more than 5 minutes at any time prior to step (ii) of the method. The active composition includes proteins which are known to be affected by temperature. For example at a temperature of greater than 71° C., proteins may start to break down; and heat denaturization of proteins may be irreversible at greater than about 80° C.

Prior to step (ii), said broth or part thereof may include a relatively high proportion of native proteins. This may be illustrated by SDS-PAGE. The ratio of native proteins to non-native proteins may be greater than 1 or greater than 2.

In the context of the present specification, a reference to a “dry matter basis” refers to an assessment of a material made ignoring any water which is present within the material assessed.

Said solid product produced in step (ii) suitably includes, on a dry matter basis, at least 10 wt % (preferably at least 30 wt %, more preferably at least 70 wt %, especially at least 90 wt %) of the total wt % of solids on a dry matter basis included in said fermentation broth from which the active composition is derived. In some, preferred embodiments, greater than 95 wt % or greater than 99 wt %) of solids in said fermentation broth are incorporated into said solid product.

Said filamentous fungus may be grown in a fermenter in a continuous or batch aerobic fermentation. Said broth selected in step (i) from which water is removed in step (ii) may (optionally, ignoring the amount of any water included) have the same composition as a fermentation broth in a fermenter upstream of a position of removal of the fermentation broth selected in step (i), from the fermenter. Such a fermentation broth is hereby referred to as “Whole Fermentation Broth” or WFB. Alternatively, said broth selected in step (i) (herein referred to as “RNA Reduced Fermentation Broth” or “RRFB”) from which water is removed in step (ii) may have a lower level of RNA compared to that in the fermentation broth in a fermenter from which the RRFB is derived.

A part of said broth from which water is removed in step (ii) may comprise a part of said WFB or a part of said RRFB. For example, said part may comprise a part produced by centrifugation of the WFB or RRFB. The part may comprise a component produced in a treatment process which does not involve filtration. The process may involve separation based on relative densities of components in a mixture (e.g. a centrifugation process). Said treatment process may yield a solid deposit or a supernatant. A part which is a deposit produced in said treatment process (e.g. by centrifugation) is referred to as “Whole Fermentation Broth-Deposit” or “WFB-D”, when it is derived from the WFB; or is referred to as “RNA Reduced Fermentation Broth-Deposit” or “RRFB-D”, when it is derived from the RRFB. A part which is a supernatant produced in said treatment process (e.g. by centrifugation) is referred to as “Whole Fermentation Broth-Supernatant” or “WFB-S”, when it is derived from the WFB; or is referred to as “RNA Reduced Fermentation Broth-Supernatant” or “RRFB-S”, when it is derived from the RRFB.

Preferably, the broth or part from which water is removed in step (ii) have a Nominal Molecular Weight Limit (NMWL) (e.g. as described at merckmillipore.com) of greater than 100 kDa, preferably greater than 120 kDa and, more preferably, greater than 150 kDa. Thus, the broth or part from which water is removed in step (ii) may include moieties which will not pass through a 120 kDa and/or a 150 kDa ultrafiltration membrane.

Said WFB and/or said RRFB suitably include native proteins. Preferably, the ratio of native proteins to non-native proteins in said WFB and/or said RRFB is greater than 1 or greater than 2.

In step (ii) of the method water is removed from the broth or a part thereof. The method may comprise removing at least 50 wt %, a least 80 wt %, at least 90 wt % of the water in the broth or part thereof.

In step (ii), said broth or part thereof is preferably not subjected to a temperature of greater than 60° C. and/or said broth or part thereof does not attain a temperature of greater than 60° C.

Step (ii) may comprise a process which includes application of heat to said broth or a part thereof or may comprise a lower temperature process. For example, water may be removed by drum drying. Alternatively, step (ii) may comprise a low temperature, for example freezing and/or freeze-drying process, for example at a temperature of less than −15° C.

In an embodiment (A), said broth or part thereof treated in step (ii) is selected from said WFB, WFB-D or WFB-S. Preferably, said WFB, WFB-D and WFB-S are not subjected to high temperature prior to step (ii). For example, prior to step (ii), said WFB, WFB-D or WFB-S are subjected to a temperature which is no greater than 50° C., preferably no greater than 40° C., more preferably no greater that 35° C. For example, after selection of the broth in step (i), the broth may be held at no greater than ambient temperature prior to step (ii). This may optimise the level of active proteins in the WFB, WFB-D or WFB-S and the activity of active components (e.g. proteins) in the active composition produced. Advantageously, said WFB, WFB-D or WFB-S may include little, if any, denaturaition of native proteins therein. Thus, the ratio of the wt % of native proteins divided by the wt % of denatured proteins may be at least 100, at least 1000, or essentially infinite.

Preparation of said WFB, WFB-D and said WFB-S suitably does not involve a treatment which reduces the level of RNA in the WFB, WFB-D or WFB-S or is intended to do. Thus, said WFB, at least, includes a level of RNA which is substantially the same as that in broth produced in the fermenter from which the WFB is selected. Said WFB-D and WFB-S also include filamentous fungus having levels of RNA based on the level in the WFB from which the WFB-D and WFB-S may be derived. For example, the sum of the level of RNA in the WFB-D and WFB-S may be substantially the same as the level of RNA in the WFB and/or in the broth produced in the fermenter.

Said WFB may include at least 3 wt %, at least 5 wt %, or at least 6 wt %, of RNA on a dry matter basis.

Said WFB-D may include at least 3 wt %, at least 5 wt %, or at least 6 wt %, of RNA on a dry matter basis.

Preferably, in said embodiment (A), said broth or part thereof is selected from WFB and WFB-D. To minimise downstream treatment of the broth, the broth treated in step (ii) is preferably said WFB. Use of the WFB may also maximise the amount of solid product produced in step (ii) per weight of fermentation broth selected in step (i).

In an embodiment (B), said broth or part thereof treated in step (ii) is selected from RRFB, RRFB-D or RRFB-S. Preferably, prior to step (ii), said RRFB, RRFB-D or RRFB-S are subjected to a temperature which is no greater than 80° C., preferably no greater than 70° C. Controlling the maximum temperature as described, may optimise the level of active proteins in the RRFB, RRFB-D or RRFB-S and the activity of active components (e.g. proteins) in the active composition produced.

Preparation of said RRFB, RRFB-D and said RRFB-S suitably involves a treatment which reduces the level of RNA in the RRFB, RRFB-D or RRFB-S or is intended to do. Thus, said RRFB, at least, includes a level of RNA which is less than in broth produced in the fermenter from which the RRFB is derived.

Said RRFB may include less than 6 wt %, less than 5 wt %, or less than 3 wt %, of RNA on a dry matter basis. Said RRFB may include at least 0.5 wt % or at least 1 wt % RNA.

Said RRFB-D may include less than 6 wt %, less than 5 wt %, or less than 3 wt %, of RNA on a dry matter basis. Said RRFB-D may include at least 0.5 wt % or at least 1 wt % RNA.

Preferably, in embodiment (B), said broth or part thereof is selected from RRFB and RRFB-D. Use of the RRFB may also maximise the amount of solid product produced in step (ii) per weight of fermentation broth selected in step (i).

The active composition of the first aspect is believed to be novel and the invention extends, in a second aspect, to an active composition produced in said method of the first aspect per se.

According to a third aspect of the invention, there is provided an active composition comprising:
(a) solid product which is a fermentation broth comprising a filamentous fungus, wherein the amount of water in the fermentation broth has been reduced to less than 10 wt %, preferably to less than 5 wt %; and/or
(b) solid product which is a part of a fermentation broth comprising a filamentous fungus, wherein the amount of water in said part has been reduced to less than 10 wt %, preferably less than 5 wt %; and/or
(c) solid product comprising a filamentous fungus, wherein the amount of water in said product is less 15 wt %, suitably less than 10 wt %, preferably less than 8 wt %, more preferably less than 7.5 wt %; and wherein, optionally, said solid product include at least 0.01 wt %, for example at least 1 wt %, at least 2 wt % or at least 3 wt %, water.

Said active composition may have wide-ranging uses as described herein. Said active composition may function as a viscosifier, gellator, foamer, foam stabiliser or emulsifier. The invention extends to a method according to the first aspect, wherein an active composition is produced which is a viscosifier, gellator, foamer, foam stabiliser or emulsifier. Said active composition may be used in a wide-range of food and non-food applications.

The solid product described in the preceding aspects may have the following characteristic: a 10 wt % solution of said solid product in de-ionised water has a viscosity of at least 0.1 Pa·s., or at least 0.12 Pa·s or at least 0.14 Pa·s when measured using the apparatus of Example 4 at 0.001 s⁻¹.

In said solid product, the ratio of native proteins to non-native proteins may be greater than 1 or greater than 2.

Suitably, said active composition includes at least 80 wt %, preferably at least 90 wt %, more preferably at least 95 wt %, especially at least 98 wt % or 99 wt % of said solid product.

Said filamentous fungus is preferably as described in the first aspect. Said filamentous fungus preferably comprises fungus selected from fungi imperfecti. Preferably, said filamentous fungus comprises, and preferably consists essentially of, cells of Fusarium species, especially of Fusarium venenatum A3/5.

Filamentous fungi in said active composition may comprise filaments having lengths of greater than 150 μm, preferably greater than 500 μm. Preferably, greater than 5 vol %, more preferably greater than 10 vol % of filamentous fungi in said active composition comprise filaments having lengths of greater than 150 μm. Preferably, fewer than 5 wt %, preferably substantially no, filaments in said active composition have lengths of greater than 5000 μm; and preferably fewer than 5 wt %, preferably substantially no filaments have lengths of greater than 2500 μm. Preferably, greater than 90 vol %, more preferably greater than 95 vol % of filamentous fungi in active composition comprise filaments having lengths of less than 2000 μm.

Said solid product may include less than 15 wt %, suitably less than 10 wt %, preferably less than 8 wt %, more preferably less than 7.5 wt % of water. Said solid product may include at least 0.01 wt %, for example at least 1 wt %, at least 2 wt % or at least 3 wt %, water.

Said solid product may include at least 35 wt %, preferably at least 40 wt %, protein on a dry matter basis. Said solid product may include less than 60 wt %, preferably less than 50 wt %, protein on a dry matter basis.

Said solid product may include at least 10 wt %, preferably at least 15 wt %, dietary fibre on a dry matter basis. Said solid product may include less than 25 wt %, preferably less than 20 wt %, dietary fibre on a dry matter basis.

Said solid product may include at least 8 wt %, preferably at least 10 wt %, fat on a dry matter basis. Said solid product may include less than 20 wt %, preferably less than 15 wt %, fat on a dry matter basis.

Said active composition may include less than 15 wt %, suitably less than 10 wt %, preferably less than 8 wt %, more preferably less than 7.5 wt % of water. Said active composition may include at least 0.01 wt %, for example at least 1 wt %, at least 2 wt % or at least 3 wt %, water.

Said active composition may include at least 35 wt %, preferably at least 40 wt %, protein on a dry matter basis. Said solid product may include less than 60 wt %, preferably less than 50 wt %, protein on a dry matter basis.

Said active composition may include at least 10 wt %, preferably at least 15 wt %, dietary fibre on a dry matter basis. Said solid product may include less than 25 wt %, preferably less than 20 wt %, dietary fibre on a dry matter basis.

Said active composition may include at least 8 wt %, preferably at least 10 wt %, fat on a dry matter basis. Said solid product may include less than 20 wt %, preferably less than 15 wt %, fat on a dry matter basis.

Preferably, said active composition and/or said solid product include moieties which will not pass through a 120 kDa and/or a 150 kDa ultrafiltration membrane.

Said active composition and/or said solid product preferably includes native proteins.

Said active composition and/or said solid product may include at least 3 wt %, at least 5 wt %, or at least 6 wt %, of RNA on a dry matter basis.

The invention extends to a method of:
(i) producing an emulsion in a material;
(ii) increasing the viscosity of a material; and/or
(iii) increasing gelation in a material;
the method comprising contacting the material with an active composition as described herein.

The material may be a food or a non-food. Where it is a food, the food may include a filamentous fungus (eg mycoprotein) as described herein in addition to said active composition.

The invention extends to the use of an active material as described herein in:
(i) foaming in a material;
(ii) emulsification in a material;
(iii) increasing the viscosity in a material; and/or
(iv) in gelation in a material.

The material may be a food or a non-food. Where it is a food, the food may include a filamentous fungus (eg mycoprotein) as described herein in addition to said active composition.

Any feature of any aspect of any invention or embodiment described herein may be combined with any other invention described herein mutatis mutandis.

Specific embodiments of the invention will now be described, by way of example, with reference to the accompanying drawings in which:

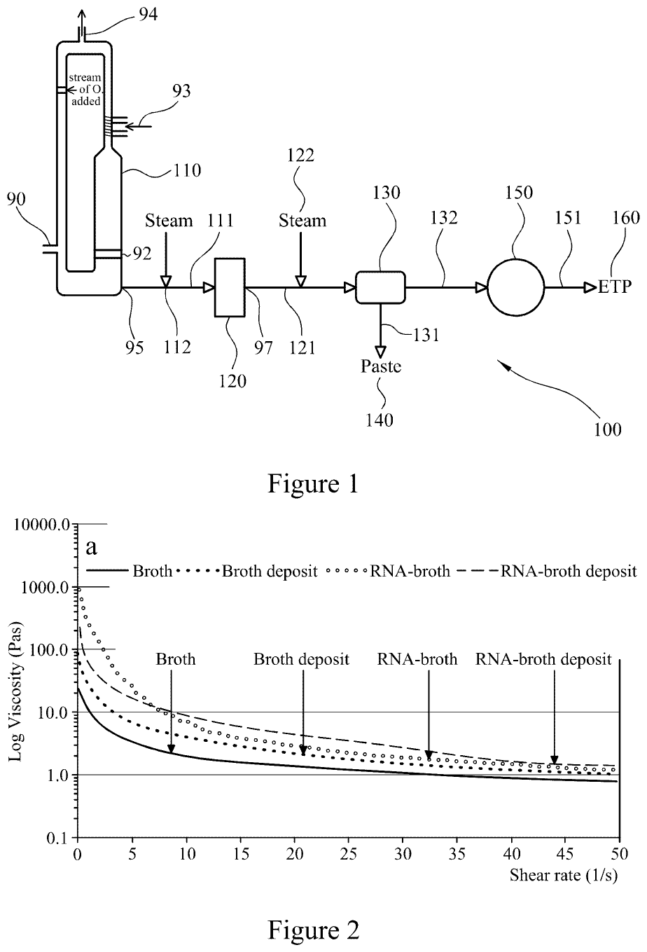

FIG. 1 is a schematic diagram showing a process for producing mycoprotein paste with reduced RNA levels by direct steam injection;

FIG. 2 is a graph showing viscosity profiles during shear rate increase of samples;

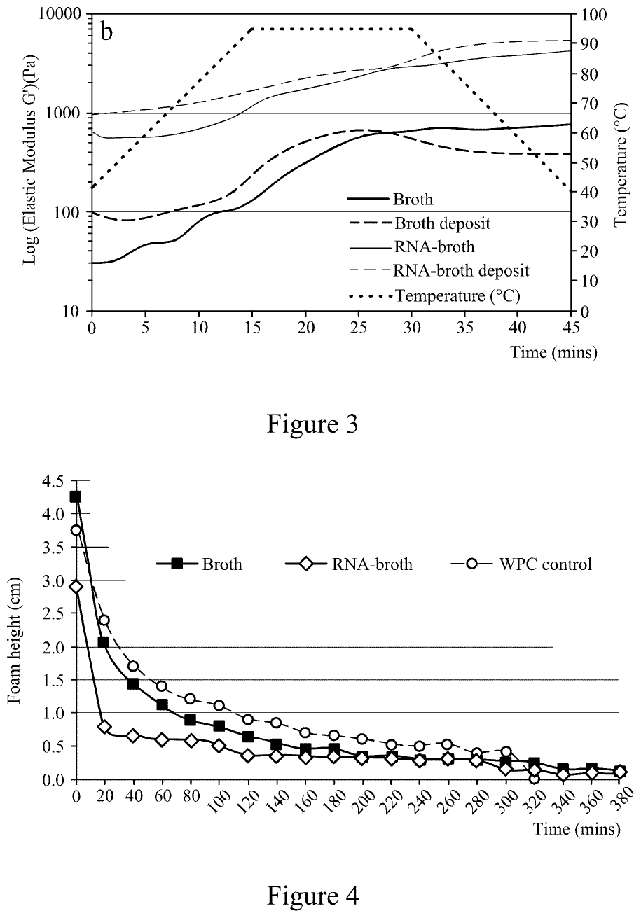

FIG. 3 is a graph showing gelation profiles (elastic modulus G′) of samples;

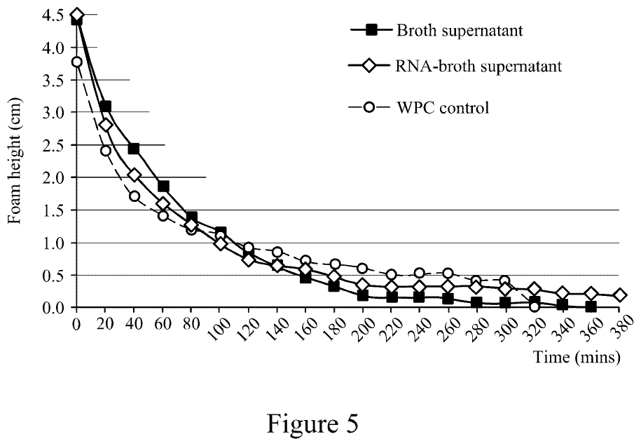

FIGS. 4 and 5 are graphs showing foaming ability and foam stability profiles of samples.

The following materials are referred to hereinafter.

Mycoprotein paste—refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852). It typically comprises about 23-25 wt % solids (the balance being water) made up of non-viable RNA reduced fungal hyphae of approximately 400-750 μm length, 3-5 μm in diameter and a branching frequency of 2-3 tips per hyphal length.

L87-Lacprodan 87, a commercial whey protein concentrate product, obtained from Arla, Denmark. It includes 87% protein and is a recognised standard whey protein isolate for evaluation of protein functionality. Hereinafter, it is referred to as whey protein concentrate (WPC) and was used as a control for all functionality tests.

Unless otherwise stated herein, particle size analysis is undertaken by laser diffraction, for example using a Beckman Coulter particle sizer.

### Example 1—Preparation of Mycoprotein Paste

Referring to FIG. 1, a commercially-used process for producing a mycoprotein paste involves growing a fungal culture in a pressure cycle fermenter 110 at 27° C. in the presence of a growth medium. The growth medium which may include glucose, biotin and minerals is introduced into the fermenter via inlet 90 and compressed gases (ammonia and air) are introduced at position 92. The fermenter includes cooling coils 93 for maintaining the temperature of the fluid in the fermenter at about 30° C. and a gas outlet 94 via which carbon dioxide exits the fermenter.

The culture broth produced passes from the fermenter 110 via an outlet at position 95 into a conduit 111 which delivers broth into an RNA reduction vessel 120. Steam (at 7 barg) and 160° C.) is injected into the culture broth via a steam injection port 112 in the conduit 111. Steam injection raises the temperature of the culture broth to 60-70° C. Steam injection is performed to reduce the RNA content of the final mycoprotein paste 140.

The RNA reduction vessel 120 is a continuously stirred tank reactor. The culture broth is held in the RNA reduction vessel 120 at the RNA reduction temperature for at least 30 minutes. The culture broth then passes from the RNA reduction vessel 120 to centrifuges 130 via a conduit 121. Steam is injected into the culture broth via a steam injection port 122 in the conduit 121. This injection of steam increases the temperature of the culture broth to 80-90° C. for hygienic purposes. The centrifuges 130 are run at 5000 g for a period of time. The centrifuges 130 separate the mycoprotein paste 140 and waste liquid centrate. The mycoprotein paste leaves the centrifuges 130 via conduit 131. The waste liquid centrate contains RNA and digestion products of RNA that have passed out of the fungal cells into the surrounding aqueous media. The waste liquid centrate, which at this stage has a temperature of 80-90° C., passes through conduit 132 to a cooler 150 in which it is cooled to 30° C. It then travels through conduit 151 to an effluent treatment plant (ETP) 160 for disposal. The final mycoprotein paste 140 has a nucleic acid content of less than 2% on a dry weight basis.

### Example 2—Preparation of Samples

Fermentation broth, referred to as Broth 1, was collected at position 95 in FIG. 1.

RNA-reduced broth (Broth 2) was collected at position 97, immediately downstream of RNA reduction vessel 120, but upstream of steam injection port 122.

Broth 1 and Broth 2 were frozen, at −20° C. with all the water included, until subsequently assessed.

Following thawing, the broths were optionally centrifuged using an AVANTI J-265 centrifuge (Beckman Coulter, UK) to separate residual solids. Samples of the whole broth streams (ie Broth 1 and Broth 2), centrifugation deposits (named deposits) (ie deposits produced after centrifugation of Broth 1 and Broth 2) and centrifugation supernatants (ie supernatants produced after centrifugation of Broth 1 and Broth 2) were freeze-dried in a Super Modulyo unit (Edwards, UK) to produce solid products including about 5 wt % water.

The samples produced are summarised in the table below:

| | | | Freeze dried |
|---|---|---|---|
| | | Freeze dried solid | supernatant |
| | | deposit collected | collected after |
| | Freeze dried whole | after centrifugation | centrifugation of |
| | broth stream | of broth | broth |
| Source | Sample reference | | |
| Broth 1 | Sample A | Sample B | Sample C |
| Broth 2 | Sample D | Sample E | Sample F |

### Example 3—Determination of Nitrogen Content of Samples

Since a large number of molecules present in the samples contain nitrogen, including fungal cell membrane and cell wall constituents such as phospholipids, glycosphingolipids, sphingomyelins, chitin, chitosan and proteins the nitrogen-containing material (NCM) content was measured using the Kjeldahl method described in Lynch J M, Barbano D M, Fleming J R (1998) Indirect and direct determination of the casein content of milk by Kjeldahl nitrogen analysis: collaborative study. Journal of AOAC International 81:763-774. The results were used to provide a guideline for preparation of standardised sample quantities for functional tests described herein.

Samples (0.1 g) of the six dried powders (Samples A to F) were digested in concentrated sulphuric acid (92%) using a Kjeltec Basic Digestion Unit 20 (Foss, UK) at 440° C. in the presence of a selenium catalyst. Distillation of the digested samples into boric acid was carried out using a Tecator Kjeltec 8100 Manual Distillation Unit (Foss, UK). The distilled samples were then titrated using 0.1 N hydrochloric acid. The % nitrogen was calculated using the following formula:

$$\% Nitrogen = \frac{Titration volume ( ml ) \times 14.007}{Sample weight ( g ) \times 100}$$

The % nitrogen obtained was then multiplied by a general conversion factor of 6.25. The experiment was repeated three times, with three replicates of each sample analysed for each experiment.

### Example 4—Assessment of Rheological Properties of Samples

Viscosity and gelation measurements were performed using a Bohlin Gemini controlled stress rheometer (Malvern Instruments, UK) using cone-and-plate geometry. 10% w/w NCM solutions of Samples A to F were prepared in deionised water and stirred for two hours. The commercial whey protein concentrate (WPC) product Lacprodan 87 (Arla, Denmark) was used as control. The WPC rheological control was prepared to match the solid content of a 10% NCM broth solution (18% solids) to account for the possible influence of solid content on viscosity. Experiments were repeated three times, with three replicates of each sample analysed for each experiment. Viscosity measurements were performed using a 4°/40 mm cone (gap 150 μm) at 20° C. The instantaneous viscosity (Pa·s) was measured through a shear rate increase from 0.001 s⁻¹ to 50 s⁻¹. Prior to gelation tests, the linear viscoelasticity region of each sample was determined via oscillatory measurements of elastic and viscous moduli (G′ and G″) carried out at 1 Hz over a strain amplitude sweep ranging from 0.00005 to 50. Gelation profiles were assessed via small-amplitude oscillatory measurements at 1 Hz using a 2°/40 mm cone (gap 70 μm) with the applied strain chosen from within the linear viscoelastic region for each sample. The elastic and viscous moduli (G′ and G″) were measured through a temperature sweep test ranging from 40 to 90° C. in up-down mode (15-minute up-sweep, 15-minute down-sweep).

### Example 5—Assessment of Foaming Properties of Samples

The stability of foams produced using Samples A to F was assessed. 15 g of solutions of 1% w/w NCM of the samples and the WPC control were prepared in 50 ml glass beakers and stirred for one hour. The solutions were frothed for 1 minute using a handheld whisk-type frother (Aerolatte, UK). The height of the resulting foam was measured immediately after whisking and every 10 mins until collapse of the foam. The foaming ability was expressed as the initial height of the foam, while the foam stability was determined as the time needed for the foam to fully collapse. The experiments were repeated four times, with three replicates of each sample analysed.

### Example 6—Assessment of Emulsifying Properties

Emulsifying activity index (EAI), emulsion stability index (ESI) and oil droplet size distribution measurements were carried out to characterise oil-in-water emulsions prepared from Samples A to F and the WPC control. EAI and ESI were determined according to Ogunwolu (see Ogunwolu S O, Henshaw F O, Mock H P, Santros A (2009) Functional properties of protein concentrates and isolates produced from cashew (Anacardium occidentale L.) nut. Food Chemistry 115:852-858) with some modifications. 22.5 g of 1% w/w NCM solutions of the samples and WPC were mixed with 7.5 g of sunflower oil (to obtain a 3:1 phase ratio) and the mixture was high-speed homogenised for 1 minutes using an Ultra Turrax high shear mixer (IKA, UK) to produce oil-in-water emulsions. 50 μl of the emulsion were pipetted from the bottom of the vial and suspended in 5 ml of 0.1% (w/v) SDS solution. This step was carried out immediately after emulsification then after 10 minutes. Absorbance of the diluted emulsions was measured at 500 nm using a Genesys 6 UV/Vis spectrophotometer (Thermo Electron Corporation, USA). The ability of the protein to form an emulsion (emulsifying activity index, EAI) and the emulsion stability index (ESI) were calculated using the following formulae:

$$EAI ( {m}^{2} / g ) = \frac{2 \times T \times {A}_{0} \times dilutionfactor}{C \times ∅ \times 1000} \qquad ESI ( min ) = \frac{{A}_{0}}{{A}_{0} - {A}_{10}} \times Δ t$$

where T=2.303, A₀=apparent absorbance at 0 minutes, dilution factor=100, C=weight per unit volume (g/mL), Ø=oil volumetric fraction (0.25), A₁₀=apparent absorbance after 10 minutes, Δt=10 minutes. Experiments were repeated three times, with three replicates of each sample analysed.

The average oil droplet size distribution of the emulsions (D[3,2], surface weighted mean) were measured using a Mastersizer 2000 (Malvern Instruments Ltd., UK). The refractive index of oil droplets was set at 1.474 (corresponding to sunflower oil) and the laser obscuration was adjusted to 10% obscuration. Experiment were repeated three times, with three replicates of each sample analysed.

Results of tests undertaken, and a discussion thereof are referred to below.

### (a) Characterisation of the Different Samples

The whole streams and centrifugation deposits of the broth and RNA-broth showed similar nitrogen-containing material (NCM) contents: 55.5% (broth-Sample A), 52.2% (RNA-broth-Sample D), 57.8% (broth deposit-Sample B) and 56.2% (RNA-broth deposit-Sample E). The NCM contents of the RNA-broth (Sample F) 45.4%) was higher than the broth supernatant (Sample C) (40.8%), which confirmed the diffusion of soluble nitrogen-containing material through the cell wall as a result of the RNA reduction of the fermented broth.

### (b) Rheological Properties

As shown in FIG. 2, 10% w/w NCM solutions of the broth (Sample A), broth deposit (Sample B), RNA-broth (Sample D) and RNA-broth deposit (Sample E) proved significantly more viscous than the WPC control (which displayed viscosity profiles below 0.1 Pa·s, results not shown in FIG. 2). The RNA-broth deposit (Sample E) showed the highest viscosity. It is believed the concentrations of fungal filaments in the broth (Sample A) and RNA-broth (Sample D) samples contributed to their high viscosity.

Unheated 10% w/w NCM solutions of the broth (Sample A), broth deposit (Sample B), RNA-broth (Sample D) and RNA-broth deposit (Sample E) proved significantly more viscoelastic than the WPC control (which displayed an initial elasticity of 0.13 Pa), believed to be due to the presence of fungal filaments in these samples, as shown in FIG. 3.

The broth (Sample A), broth deposit (Sample B), RNA-broth (Sample D) and RNA-broth deposit (Sample E) all displayed a high level of gel-like behaviour as illustrated in FIG. 3. In this regard, the gels obtained with the broth, broth deposit, RNA-broth and RNA-broth deposit proved more viscoelastic than WPC gels (which displayed a final elasticity of 1,364 Pa (result not shown in FIG. 3). Similar to their unheated solutions, the RNA-broth gels (whole stream and deposit) proved more viscoelastic than their broth counterparts, and the gels prepared with the deposits (broth and RNA-broth) proved more viscoelastic than the ones prepared with their whole stream counterparts which is believed to be due to their different concentrations of fungal filaments.

### (c) Foaming Properties

All the samples displayed higher foaming abilities (ie the first data point which represents the initial foam level) than the WPC control, except for the RNA-broth solution (Sample D), as illustrated in FIGS. 4 and 5.

Foams produced with all of Samples A to F proved more stable than the WPC foams. Foams prepared with broth (Sample A), RNA-broth (Sample D) and RNA-broth supernatant (Sample F) displayed the highest stabilities.

It is believed that the lower foaming ability but high foam stability reported for the RNA-broth could be due to its high entanglement of fungal filaments following the heat-shock RNA-reduction process and its associated high viscosity. The high stability reported for broth and RNA-broth foams could then result from the concentration of fungal filaments in these samples while the presence of foam-positive molecules in the RNA-broth supernatants could have contributed to their high foaming ability and stability. In particular, the concentrations of the cerato-platanin protein in the RNA-broth supernatant foams proved higher than in the other samples which may contribute to the high stabilities of foams prepared. A range of metabolites, including cell wall chitin and chitosan and cell membrane phospholipids, were reported in higher concentrations following the RNA-reducing heat-shock treatment and possibly contributed to the emulsifying properties of the RNA-broth samples.

### (d) Emulsifying Properties

Emulsions prepared with the two supernatant samples (broth (Sample C), RNA-broth (Sample F)), as well as the broth (Sample A) and broth deposit (Sample B), displayed similar or higher emulsifying activity index (EAI) than the ones prepared with WPC. The emulsifying stability index (ESI) obtained for broth (Sample A) and broth supernatant (Sample C) emulsions also proved similar or higher than those obtained with WPC. The emulsions prepared with all the samples displayed similar or smaller mean oil droplet sizes (D[3,2], surface weighted mean values) than WPC emulsions. Emulsions prepared with broth (Sample A), broth deposit (Sample B), RNA-broth (Sample D) and RNA-broth deposit (Sample E) showed a network of fungal filaments surrounding the oil droplets.

The RNA-broth deposit emulsions (Sample E), showed the lowest EAI but the highest ESI of all samples.

Overall, the results illustrate the potential of using the fermentation products described to produce functional ingredients for the food (and other) industries. Highlights include:
The broth (Sample A), broth deposit (Sample B), RNA-broth (Sample D) and RNA-broth deposit (Sample E), showed high potential as thickening and gelling agents. Solutions of these samples displayed high viscosity, whilst hydrogels prepared with these solutions proved highly viscoelastic.
The broth (Sample A), broth deposit (Sample B), RNA-broth (Sample D) and RNA-broth deposit (Sample E), showed high foam stability so the materials are potential foaming agents for industry.
The broth (Sample A), broth deposit (Sample B), RNA-broth (Sample D) and RNA-broth deposit (Sample E), have high ESI and low oil droplet size which reinforces the materials as potential foaming agents for industry.
RNA-broth (Sample D) and RNA-broth deposit (Sample E) include higher concentrations of:
(i) cerato-platanin protein (which are believed to contribute to foaming and emulsifying properties);
(ii) nucleoporin NSP1 protein (believed to contribute to gelling properties);
(iii) chitin and chitosan (believed to contribute to thickening, gelling, foaming and emulsifying properties);
(iv) nucleobases including guanine (believed to contribute to thickening and gelling properties);
(v) entangled fungal filaments;

In addition, RNA-broth (Sample D) and RNA-broth deposit (Sample E) have higher viscoelasticity in solution and resulting gels than the broth (Sample A) and broth deposit (Sample B) which may be due to rheological properties of entangled fungal filaments and/or potential contribution of higher nucleoporin NSP1, chitin, chitosan and nucleobases including guanine.

The RNA-broth supernatant (Sample F) showed potential as a foaming agent with high foaming ability and stability.

As an alternative to the broths being freeze dried as described, they may readily be drum dried.

Thus, it should be appreciated that the broths described may be treated and used to produce potential ingredients which have a range of advantageous properties. For example, ingredients could be produced which can be used as emulsifiers which could replace existing chemical surfactants.

Additionally, ingredients could be used as viscosifiers or for stabilising foamed products.

Advantageously, the ingredients described are “natural” and also vegan. They could therefore be used in “clean label” foodstuffs.

The ingredients described may have wide-ranging non-food applications, for example, in household goods (e.g. soaps, detergent, fabric conditioner), in cosmetics (e.g. personal care, skin care, cleansers, deodorants, hair care, perfumery, sunscreens, moisturizers), in industrial goods (e.g. inks, lubricants, anti-fogging, liquids, adhesives) and in pharmaceutical, fungicides and herbicides.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of producing an active composition, the method comprising:
(i) selecting a fermentation broth comprising a filamentous fungus; and
(ii) removing water from said broth or a part thereof to produce a solid product, wherein said solid product is an active composition.

2. The method according to claim 1, wherein said filamentous fungus comprises a Fusarium species, especially of Fusarium venenatum A3/5.

3. The method according to claim 1, wherein said filamentous fungus in said broth comprises:
filaments having lengths of greater than 150 μm; and/or
greater than 5 vol %, more preferably greater than 10 vol %, of filamentous fungus in said broth comprise filaments having lengths of greater than 150 μm.

4. The method according to claim 1, wherein, in said solid product, said filamentous fungus comprises filaments having lengths of greater than 150 μm.

5. The method according to claim 1, wherein said solid product includes less than 15 wt % of water; and/or said solid product includes at least 0.01 wt % water.

6. The method according to claim 1, wherein said solid product includes at least 35 wt % protein on a dry matter basis; and/or includes less than 60 wt % protein on a dry matter basis.

7. The method according to claim 1, wherein said solid product includes at least 10 wt % dietary fibre on a dry matter basis; and/or said solid product includes less than 25 wt % dietary fibre on a dry matter basis.

8. The method according to claim 1, wherein said solid product includes at least 8 wt % fat on a dry matter basis; and/or said solid product includes less than 20 wt % fat on a dry matter basis.

9. The method according to claim 1, wherein said solid product produced in step (ii) includes, on a dry matter basis, at least 10 wt % of the total wt % of solids on a dry matter basis included in said fermentation broth from which the active composition is derived.

10. The method according to claim 1, wherein the broth or part from which water is removed in step (ii) has a Nominal Molecular Weight Limit (NMWL) of greater than 100 kDa, preferably greater than 150 kDa; and/or the broth or part from which water is removed in step (ii) includes moieties which will not pass through a 120 kDa and/or a 150 kDa ultrafiltration membrane.

11. The method according to claim 1, wherein, in step (ii), said broth or part thereof is not subjected to a temperature of greater than 60° C. and/or said broth or part thereof does not attain a temperature of greater than 60° C.

12. The method according to claim 1, wherein said broth selected in step (i) from which water is removed in step (ii) has the same composition as a fermentation broth in a fermenter upstream of a position of removal of the fermentation broth selected in step (i), from the fermenter, said fermentation broth being hereby referred to as “Whole Fermentation Broth” or WFB; or said broth selected in step (i), referred to as “RNA Reduced Fermentation Broth” or “RRFB” from which water is removed in step (ii), has a lower level of RNA compared to that in the fermentation broth in a fermenter from which the RRFB is derived.

13. The method according to claim 12, wherein a part of said broth from which water is removed in step (ii) comprises a part of said WFB or a part of said RRFB, wherein a part which is a deposit produced in a treatment process is referred to as “Whole Fermentation Broth-Deposit” or “WFB-D”, when it is derived from the WFB; or is referred to as “RNA Reduced Fermentation Broth-Deposit” or “RRFB-D”, when it is derived from the RRFB.

14. The method according to claim 12, wherein said WFB and/or said RRFB include native proteins; and/or wherein said WFB includes at least 3 wt %, at least 5 wt %, or at least 6 wt %, of RNA on a dry matter basis; and/or said WFB-D includes at least 3 wt %, at least 5 wt %, or at least 6 wt %, of RNA on a dry matter basis.

15. An active composition produced in said method of claim 1.

16. An active composition comprising:
(a) solid product which is a fermentation broth comprising a filamentous fungus, wherein the amount of water in the fermentation broth has been reduced to less than 10 wt %; and/or
(b) solid product which is a part of a fermentation broth comprising a filamentous fungus, wherein the amount of water in said part has been reduced to less than 10 wt %; and/or
(c) solid product comprising a filamentous fungus, wherein the amount of water in said product is less 15 wt %; and wherein, optionally, said solid product include at least 0.01 wt % water.

17. The active composition according to claim 16, wherein a 10 wt % solution of said solid product in de-ionised water has a viscosity of at least 0.1 Pa·s or at least 0.12 Pa·s or at least 0.14 Pa·s when measured using the apparatus of Example 4 at 0.001 s⁻¹.

18. The active composition according to claim 15, wherein, in said solid product, the ratio of native proteins to non-native proteins is greater than 1.

19. The active composition according to claim 1, wherein said filamentous fungus is a Fusarium specie.

20. The active composition according to claim 16, wherein filamentous fungus in said active composition comprises filaments having lengths of greater than 150 μm; and/or greater than 5 vol % of filamentous fungus in said active composition comprises filaments having lengths of greater than 150 μm.

21. The active composition according to claim 16, wherein said solid product includes less than 15 wt % of water; and/or includes at least 35 wt % e protein on a dry matter basis; and/or includes less than 60 wt % protein on a dry matter basis; and/or includes at least 10 wt % dietary fibre on a dry matter basis; and/or includes less than 25 wt % dietary fibre on a dry matter basis; and/or includes at least 8 wt % fat on a dry matter basis; and/or includes less than 20 wt % fat on a dry matter basis.

22. The active composition according to claim 16, wherein said active composition and/or said solid product include moieties which will not pass through a 120 kDa and/or a 150 kDa ultrafiltration membrane.

23. The active composition according to claim 16, wherein said active composition and/or said solid product includes native proteins.

24. The active composition according to claim 16, wherein said active composition and/or said solid product include at least 3 wt % or at least 6 wt % of RNA on a dry matter basis.

25. A method of:
(i) producing an emulsion in a material;
(ii) increasing the viscosity of a material; and/or
(iii) increasing gelation in a material;
the method comprising contacting the material with an active composition as described in any of claim 15.

26. The method according to claim 25, wherein said material is a food or a non-food.

27. The use of an active material of claim 16, for:
(i) foaming in a material;
(ii) emulsification in a material;
(iii) increasing the viscosity in a material; and/or
(iv) gelation in a material.
